(19) United States
(12) Patent Application Publication
(10) Pub. No.: US 2006/0009644 A1
(43) Pub. Date: Jan. 12, 2006

(54) Novel crystalline forms of sodium 1,2-benzisoxazole-3-methanesulfonate, processes of preparing same and use thereof in the synthesis of zonisamide

(76) Inventors: Vladimir Naddaka, Lod (IL); Itai Adin, Beer-Sheva (IL); Eyal Klopfer, Tel Aviv (IL); Oded Arad, Rechovot (IL); Joseph Kaspi, Givatayim (IL)

Correspondence Address: Martin D. Moynihan;PRTSI, Inc., P.O. Box 16446, Arlington, VA, 22215, US

(21) Appl. No.: 11/153,403

(22) Filed: Jun. 16, 2005

Related U.S. Application Data
(60) Provisional application No. 60/582,086, filed on Jun. 24, 2004.
(60) Provisional application No. 60/580,360, filed on Jun. 18, 2004.
(60) Provisional application No. 60/622,009, filed on Oct. 27, 2004.

(51) Int. Cl. C07D 261/20

(52) U.S. Cl. 548/241

(57) ABSTRACT

Disclosed is a process of preparing 1,2-benzisoxazole-3-methanesulfonamide (zonisamide). Also disclosed is a method of dehydrating sodium 1,2-benzisoxazole-3-methanesulfonate, a compound useful in the preparation of 1,2-bisoxazole-3-methanesulfonamide (zonisamide) as well as new crystalline forms of sodium 1,2-benzisoxazole-3-methanesulfonate.

The present application gains the benefit of U.S. Provisional Patent Application No. 60/582,086 filed on Jun. 24, 2004, U.S. Provisional Patent Application No. 60/580,360 filed on Jun. 18, 2004, and U.S. Provisional Patent Application No. 60/622,009 filed on Oct. 27, 2004, the teachings of which are incorporated herein in their entirety.

### FIELD AND BACKGROUND OF THE INVENTION

The present invention is of novel crystalline forms of sodium 1,2-benzisoxazole-3-methanesulfonate (also referred to herein, interchangeably as BIOS-Na), processes for preparing same and for dehydrating same and of use thereof in the preparation of zonisamide.

Zonisamide (3-(sulfamoylmethyl)-1,2-benzisoxazole or 1,2-benzisoxazole-3-methanesulfonamide) is represented by structural formula I.

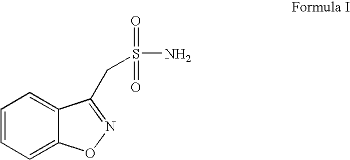

Zonisamide is used as an anti-epileptic agent, exhibiting anti-convulsive and anti-neurotoxic effects.

In U.S. Pat. No. 4,172,896, to Uno H. et al. (assigned to Dainippon Pharmaceutical Co.) is described a process for the preparation of zonisamide by the route described in Scheme 1 below.

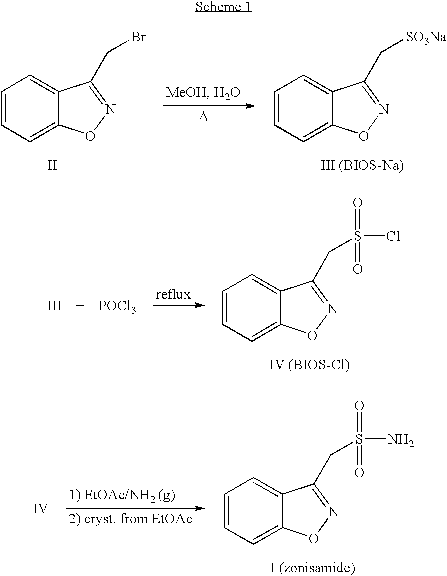

As shown in Scheme 1, 3-bromomethyl-1,2-benzisoxazole (Compound II) is converted to sodium 1,2-benzisoxazole-3-methanesulfonate (Compound III, hereinafter BIOS-Na) by reaction with sodium sulfite in a methanol-water mixture at 50° C. In the following step, BIOS-Na (Compound III) is converted to 1,2-benzisoxazole-3-methanesulfonyl chloride (Compound IV, hereinafter BIOS-Cl) by reaction with a large excess of phosphorus oxychloride. In the last step BIOS-Cl is treated with gaseous ammonia in ethyl acetate to yield zonisamide (Compound I ). Zonisamide is thereafter recrystallized from ethyl acetate.

While practicing the process described in U.S. Pat. No. 4,172,896, it has been found that the BIOS-Na (Compound III) is obtained as a monohydrate having a 7% water content by weight (see, Reference Example 1 in the Examples section that follows).

When a BIOS-Na monohydrate is brought in contact with a chlorinating agent (e.g., phosphorus oxychloride, phosphorous pentachloride, thionyl chloride, oxalyl chloride) as in the second step of Scheme 1, the water molecule of the BIOS-Na monohydrate reacts with the chlorinating agent releasing toxic, corrosive and environmentally damaging hydrogen chloride. Further, to compensate for the chlorinating agent that react with the water in the BIOS-Na monohydrate, excess chlorinating agent must be added. It is therefore desirable to dehydrate BIOS-Na monohydrate produced as described in Scheme 1 before contact with a chlorinating agent. However, as taught for example, in U.S. Pat. No. 6,841,683 (assigned to Teva Pharmaceutical USA, inc.) drying BIOS-Na monohydrate is time and energy consuming as it requires placing the BIOS-Na in an oven for a long time (about 48 hours). In addition, the dried BIOS-Na is hygroscopic and therefore inconvenient for use.

U.S. Pat. No. 6,841,683 and U.S. patent application Ser. No. 10/288,135 (published as U.S. 2003/0144527) by Nidam T. et al. (assigned to Teva Pharmaceutical USA, Inc.) describe five crystalline forms of BIOS-Na (Crystalline Forms I, II, III, IV and V), obtained by the reaction of 1,2-benzisoxazole-3-methanesulfonic acid hereinafter; BIOS-H) with sodium hydroxide.

Crystalline form I of BIOS-Na (a hydrate having a water content of about 7%) was prepared by exposing BIOS-Na with a water content of about 1.7% to humid laboratory air for one week followed by storage in a sealed bottle at room temperature for a period of 5 months.

Crystalline form II of BIOS-Na (having a water content of about 1.83%) was prepared by drying BIOS-Na in an oven at 80° C. for 2 days.

Crystalline form III of BIOS-Na (the water content was not reported) was prepared by treating BIOS-H in toluene with NaOH pearls followed by drying the obtained solid in an oven at 80° C. for 2 days.

Crystalline form IV (having a water content of about 2.9%) was prepared by slurrying BIOS-Na in absolute ethanol to obtain a jelly-like mass followed by solvent removal under reduced pressure.

Crystalline form V (having a water content of less than 1.5%) was prepared by drying BIOS-Na under reduced pressure at 85° C. during several days.

The high time and energy requirements render the above-described processes economically unviable on an industrial scale. Specifically, as is seen above, removing water from the BIOS-Na requires a very long period of time, generally a few days.

There is thus a widely recognized need for, and it would be highly advantageous to have, novel crystalline forms of BIOS-Na devoid of the above limitations, which could be efficiently used in the preparation of zonisamide. There is further a widely recognized need for, and it would be highly advantageous to have, novel processes for dehydrating BIOS-Na, so as to provide BIOS-Na with a low water content, devoid of the above limitations.

### SUMMARY OF THE INVENTION

The present invention provides a process for the dehydration of BIOS-Na useful for the preparation of zonisamide. The present invention also provides two novel crystalline forms of BIOS-Na that are suitable for use in the preparation of zonisamide, and processes for the preparation thereof. The present invention also provides processes of preparing zonisamide from any one of the two novel crystalline forms of BIOS-Na and/or from other suitable intermediates.

The general techniques that may lead to the discovery of a novel crystalline form of a given compound are well known to those who are skilled in the art. Such techniques include crystallization from solvents, thermal treatment, and sublimation. It is impossible to predict the experimental conditions that will produce a new crystalline form of a known compound. Rather, the search for new crystalline forms of a given compound is empirical, involving many trial and error experiments with different techniques and conditions where each individual experiment is expected to fail.

According to one aspect of the present invention there is provided a crystalline sodium 1,2-benzisoxazole-3-methanesulfonate (BIOS-Na) Form A. According to a feature of the present invention, the crystalline BIOS-Na Form A comprises at least one of the following characteristics: a powder X-ray diffraction pattern exhibiting peaks at diffraction angles of about 5.3 and 15:8±0.2 °2θ; and an infrared spectrum exhibiting absorption peaks at 3605, 3532, 1620, 1605, 1064 and 662 cm⁻¹.

According to a feature of the present invention, crystalline BIOS-Na Form A is further characterized by a powder X-ray diffraction pattern exhibiting peaks at diffraction angles of about 14.5, 21.1, 21.8 and 26.4±0.2 °2θ, or substantially as depicted in FIG. 4.

According to a feature of the present invention, crystalline BIOS-Na Form A is further characterized by an infrared spectra further exhibiting absorption peaks at 3605, 3532, 2924, 2853, 1620, 1605, 1516, 1438, 1341, 1260, 1213, 1160, 1064, 1005, 777, 748, 662, 588 and 521 cm⁻¹, or substantially as depicted in FIG. 5.

In an embodiment of the present invention, the crystalline BIOS-Na Form A has a water content lower than 1.5% by weight. In an embodiment of the present invention, the crystalline BIOS-Na Form A has a water content that ranges from about 1% to about 1.4% by weight.

According to another aspect of the present invention there is also provided a process of preparing crystalline sodium 1,2-benzisoxazole-3-methanesulfonate (BIOS-Na) Form A, the process comprising: providing a mixture including sodium 1,2-benzisoxazole-3-methanesulfonate and toluene (which in embodiments of the present invention is a suspension); refluxing the mixture (that is heating and maintaining the mixture at a boiling or reflux temperature) while removing water (preferably as an azeotrope of toluene and water); and isolating the formed BIOS-Na Form A from the mixture, thereby obtaining the crystalline BIOS-Na Form A.

In an embodiment of the present invention, the mixture comprises at least 2 ml (or, e.g., at least 5 ml or at least 7 ml or at least 10 ml) toluene for every gram sodium 1,2-benzisoxazole-3-methanesulfonate.

In an embodiment of the present invention the mixture is substantially a suspension of the sodium 1,2-benzisoxazole-3-methanesulfonate in toluene.

In an embodiment of the present invention, prior to the isolating of the BIOS-Na Form A, the mixture is cooled either by active cooling or by passively letting the mixture cool down. In an embodiment of the present invention, the mixture is cooled to a temperature lower than or equal to about 25° C.

In an embodiment of the present invention, subsequent to the isolating, the BIOS-Na Form A is dried. In an embodiment of the present invention the drying of the BIOS-Na Form A is effected at room temperature.

According to still another aspect of the present invention there is provided a use of BIOS-Na Form A in the preparation of 1,2benzisoxazole-3-methanesulfonamide (zonisamide).

According to yet another aspect of the present invention there is o provided a process of preparing 1,2-benzisoxazole-3-methanesulfonamide (zonisamide), the process comprising: providing the crystalline BIOS-Na Form A as described above; and converting the BIOS-Na Form A to zonisamide. Preferably, the BIOS-Na is converted to zonisamide as is detailed hereinunder.

According to an additional aspect of the present invention there is provided a crystalline sodium 1,2-benzisoxazole-3-methanesulfonate (BIOS-Na) Form B. According to a feature of the present invention, the crystalline BIOS-Na Form B comprises at least one of the following characteristics: a powder X-ray diffraction pattern exhibiting peaks at diffraction angles of about 4.8 and 5.3±0.2 °2θ; and an infrared spectrum exhibiting absorption peaks at 3543, 3484, 3436, 1639, 1613, 1049, 761 and 742 cm⁻¹.

According to a feature of the present invention, crystalline BIOS-Na Form B is further characterized by a powder X-ray diffraction pattern exhibiting peaks at diffraction angles of about 6.0, 14.4, 15.2, 15.7, 16.4, 21.1, 21.8 and 26.5±0.2 °2θ, or substantially as depicted in FIG. 6.

According to a feature of the present invention, crystalline BIOS-Na Form B is further characterized by an infrared spectrum further exhibiting absorption peaks at 3543, 3484, 3436, 2923, 2854, 1639, 1613, 1514, 1439, 1342, 1235, 1212, 1199, 1160, 1063, 1049, 1006, 761, 742, 669 and 589 cm⁻¹, or substantially as depicted in FIG. 7.

In an embodiment of the present invention, the crystalline BIOS-Na Form B has a water content lower than 1.5% by weight. In an embodiment of the present invention, the crystalline BIOS-Na Form B has a water content that ranges from about 1% to about 1.4% by weight.

According to yet an additional aspect of the present invention there is also provided a process of preparing crystalline sodium 1,2-benzisoxazole-3-methanesulfonate (BIOS-Na) Form B, the process comprising: providing a mixture including sodium 1,2-benzisoxazole-3-methanesulfonate and a solvent mixture including toluene and N,N-dimethylformamide (DMF) as solvent components (which in embodiments of the present invention becomes a gel upon heating); refluxing the mixture (that is heating and maintaining the mixture at a boiling or reflux temperature) while removing water (preferably as an azeotrope of toluene and water); and isolating the formed BIOS-Na Form B from the mire, thereby obtaining the crystalline BIOS-Na Form B.

According to a feature of the present invention the order of adding the mixture components is not important, and one or more of the components may be added after heating has commenced, or even during reflux.

In an embodiment of the present invention, the mixture comprises at least 2 ml (or, e.g., at least 5 ml or at least 7 ml or at least 10 ml) toluene for every gram sodium 1,2-benzisoxazole-3-methanesulfonate.

In an embodiment of the present invention, the mixture comprises at least 0.1 mole (and preferably at least 1 mole) DMF for every mole sodium 1,2-benzisoxazole-3-methanesulfonate.

In an embodiment of the present invention the mixture is substantially a suspension of the sodium 1,2-benzisoxazole-3-methanesulfonate in the solvent.

In an embodiment of the present invention, prior to the isolating of the BIOS-Na Form B, the mixture is cooled either by active cooling or by passively letting the mixture cool down. In an embodiment of the present invention, the mixture is cooled to a temperature lower than or equal to about 25° C.

In an embodiment of the present invention, subsequent to the isolating, the BIOS-Na Form B is dried. In an embodiment of the present invention the drying of the BIOS-Na Form B is effected at room temperature.

According to the teachings of the present invention there is also provided a use of BIOS-Na Form B in the preparation of 1,2-benzisoxazole-3-methanesulfonamide (zonisamide).

According to still an additional aspect of the present invention there is also provided a process of preparing 1,2-benzisoxazole-3-methanesulfonamide (zonisamide), the process comprising: providing the crystalline BIOS-Na Form B as described above; and converting the BIOS-Na Form B to zonisamide. Preferably, the BIOS-Na Form B is converted to zonisamide as is detailed hereinunder.

According to a further aspect of the present invention there is provided a process for dehydrating sodium 1,2-benzisoxazole-3-methanesulfonate (BIOS-Na), the process comprising: providing a mixture of BIOS-Na and an organic solvent (which may include one or more solvent components and where the mixture may be of any form including but not limited to a solution, slurry or suspension); and refluxing (that is heating and maintaining the mixture at a boiling or reflux temperature) the mixture while removing water therefrom (preferably as an azeotrope of at least one solvent component and water).

In an embodiment of the present invention, the solvent comprises toluene.

In an embodiment of the present invention, the mixture comprises at least 2 ml (or e.g., at least 5 ml or at least 7 ml or at least 10 ml) toluene for every gram sodium 1,2-benzisoxazole-3-methanesulfonate.

In an embodiment of the present invention, the solvent comprises N,N-dimethylformamide (DMF). In an embodiment of the present invention, the mixture comprises at least 0.1 mole (and preferably at least 1 mole) DMF for every mole sodium 1,2-benzisoxazole-3-methanesulfonate.

In an embodiment of the present invention, of the solvent comprises a mixture of toluene and DMF, possibly with minor impurities or water.

In an embodiment of the present invention, the mixture comprises at least 2 ml (or, e.g., at least 5 ml or at least 7 ml or at least 10 ml) toluene for every gram sodium 1,2-benzisoxazole-3-methanesulfonate.

As used herein, the terms “comprising” and “including” or grammatical variants thereof are to be taken as specifying the stated features, integers, steps or components but do not preclude the addition of one or more additional features integers, steps, components or groups thereof These terms encompass the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Implementation of the methods of the present invention involves performing or completing selected tasks or steps manually, automatically, or a combination thereof.

As used herein the term “mixture” describes a mixture that includes more than one substance and which can be in any form, for example, as a homogenous solution, a suspension, a dispersion, a biphasic solution and more.

As used in this application, the singular form “a”, “an” and “the” include plural references unless the context clearly dictates otherwise.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE FIGURES

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents an X-ray powder diffractogram of BIOS-Na monohydrate prepared according to the process taught in U.S. Pat. No. 4,172,896;

FIG. 2 presents a differential scanning calorimetry curve of BIOS-Na monohydrate prepared according to the process taught in U.S. Pat. No. 4,172,896;

FIG. 3 presents the results of thermal gravimetric analysis of BIOS-Na monohydrate prepared according to the process taught in U.S. Pat. No. 4,172,896;

FIG. 4 presents an X-ray powder diffractogram of BIOS-Na Form A of the present invention;

FIG. 5 presents an infrared spectrum of BIOS-Na Form A of the present invention;

FIG. 6 presents an X-ray powder diffractogram of BIOS-Na Form B of the present invention; and

FIG. 7 presents an infrared spectrum of BIOS-Na Form B of the present invention.

### DESCRIPTION OF EMBODIMENTS OF THE PRESENT INVENTION

The present invention is of novel crystalline forms of BIOS-Na; processes for the preparation thereof; and use thereof in the preparation of zonisamide. The present invention is further of a process of dehydrating BIOS-Na, so as to provide BIOS-Na with a low water content. The present invention is further of a novel process of preparing zonisamide.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As used herein, the term “about” refers to ±10%.

As discussed above, one of the difficulties encountered in the synthesis of zonisamide from BIOS-Na is the fact that prior art BIOS-Na is a hydrate and contains 7% by weight water. As water reacts with a chlorination agent, which is introduced to the BIOS-Na during the synthesis of zonisamide (see, for example Scheme 1), it is necessary to add a large excess of a chlorinating agent to compensate for the loss of chlorinating agent that reacts with the water. Prior efforts for removing water from BIOS-Na are not appropriate for industrial implementation due to the high energy and time requirements.

In a search for more efficient processes of dehydrating BIOS-Na, the present inventors have envisioned that the well-known azeotropic distillation technique for removing water from a given solvent or mixture can be used for that purpose. While reducing the present invention to practice, it was indeed found that by refluxing a mixture containing BIOS-Na, either as a monohydrate or as other hydrated form thereof, while removing water therefrom by means of an azeotrope of the water with the mixture solvent, results in substantially dehydrated BIOS-Na.

Thus, according to one aspect of the present invention there is provided a process for dehydrating BIOS-Na By “dehydrating” it is meant herein removing a significant proportion (e.g., greater than 50%, preferably greater than 60% and more preferably greater than 80%) of the water content in BIOS-Na. The dehydration process of the present invention is economical in terms of time and energy. Further, the dehydration process of the present invention allows isolation of crystalline BIOS-Na forms (vide infra) that can be isolated and stored for later use or allows the immediate use of the dehydrated product in the preparation of zonisamide, for example in accordance with the process described hereinabove in Scheme I.

The process of dehydrating sodium 1,2-benzisoxazole-3-methanesulfonate (BIOS-Na), is effected by providing a mixture of BIOS-Na and a non-aqueous solvent, e.g., an organic solvent (which may include one or more solvent components and where the mixture may be of any form including but not limited to a solution, slurry or suspension); and refluxing (that is heating and maintaining the mixture at a boiling or reflux temperature) the mixture while removing water (preferably as an azeotrope of at least one solvent component and water).

Substantially, the dehydration process, according to this aspect of the present invention, is effected by mixing BIOS-Na with a solvent comprising one or more non-aqueous solvent components, to thereby provide a mixture containing the BIOS-Na and a non-aqueous solvent, and then heating the mixture to a reflux temperature (refluxing) while removing water therefrom.

As used herein, the phrase “refluxing a mixture while removing water therefrom” in meant to describe an azeotropic distillation of the mixture, in which the water is removed from the mixture as an azeotrope of water and the solvent.

As is well known in the art, an “azeotrope” refers to a constant boiling mixture of two or more substances that behaves like a single substance, at least during a period of a distillation process in which the two substances are present in a mixture. Thus, an azeotrope exhibits either a maximum, or minimum boiling point compared to the boiling point of one of the substances.

Similarly, as used herein, the phrase “azeotropic distillation” describes a distillation procedure in which an azeotrope is removed from a mixture. As is well-recognized in the an, this phrase is commonly used to describe a distillation procedure in which an azeotrope of water and a non-aqueous, organic solvent is distilled our of a mixture.

In an embodiment of the present invention, at least 50% (or 60%, or 70%, or 80%, or 90% or 95% or 97.5% or 99%) by weight of the solvent is toluene. In an embodiment of the present invention the solvent substantially consists of toluene, possibly with minor impurities or water.

In an embodiment of the present invention, the mixture comprises at least 2 ml (or, e.g., at least 5 ml or at least, 7 ml or at least 10 ml) toluene for every gram sodium 1,2-benzisoxazole-3-methanesulfonate.

In an embodiment of the present invention, N,N-dimethylformamide (DMF) is a solvent component. In an embodiment of the present invention, the mixture comprises at least 0.1 mole (and preferably at least 1 mole) DMF for every mole sodium 1,2-benzisoxazole-3-methanesulfonate.

In an embodiment of the present invention, at least 50% (or 60%, or 70%, or 80%, or 90% or 95% or 97.5% or 99%) by weight of the solvent is toluene and/or DMF. In an embodiment of the present invention the solvent substantially consists of a mixture of toluene and DMF, possibly with minor impurities or water.

In an embodiment of the present invention, the mixture of toluene and DMF comprises at least 2 ml (or, e.g., at least 5 ml or at least 7 ml or at least 10 lm) toluene for every gram sodium 1,2-benzisoxazole-3-methanesulfonate.

Sodium 1,2-benzisoxazole-3-methanesulfonates (BIOS-Na) that can be effectively dehydrated using the process described herein include, without limitation. BIOS-Na monohydrate, a crystalline form of BIOS-Na such as those described hereinbelow and those described in, for example, U.S. Pat. No. 6,841,683, U.S. patent application Ser. No. 10/288,135 (published as U.S. 2003/0144527) by Nidam T. et al. (assigned to Teva Pharmaceutical USA, Inc.) and WO 03/020708, which are all incorporated by referent as if fully set forth herein. Thus, a BIOS-Na having a water content of, for example, from 1.5% to about 7%, can be effectively dehydrated using this process.

In an embodiment of the present invention, the BIOS-Na containing product resulting from the above-described dehydration of BIOS-Na (in embodiments of the present invention, a suspension of BIOS-Na in a solvent) is used directly in the preparation of zonisamide using any of the appropriate processes known in the art, but preferably the process of the present invention that is described hereinbelow in detail and is substantially described by Scheme I. It has been found that the reactivity of BIOS-Na dehydrated according to the dehydration process described herein, is higher than the reactivity of non-dehydrated BIOS-Na (e.g., a BIOS-Na monohydrate), and of the reactivity of BIOS-Na dehydrated according to prior art methods. For example, it has been found that when such a BIOS-Na containing product of the dehydration process, as described above, is directly used according to the process of the present invention described below, crude zonisamide is obtained with a greater than 90% yield and contains less than 1% salts of 1,2-benzisoxazole-3-methanesulfonic acid (as determined by HPLC).

In embodiments of the present invention, crystalline BIOS-Na is isolated from) the BIOS-Na containing product resulting fm the above-described dehydration of BIOS-NA. The composition of the solvent determines the resulting crystalline BIOS-Na form: When the solution includes substantially only toluene, a novel crystalline BIOS-Na, designated Form A is produced. When the solution includes a mixture of toluene and DMF, a novel crystalline BIOS-Na, designated Form B is produced. Both BIOS-Na Form A and BIOS-Na Form B can be used for the preparation of zonisamide. Either of the crystalline BIOS-Na forms is converted to BIOS-Na using any of the appropriate processes known in the art, but preferably the process of the present invention that is described hereinbelow in detail and is substantially described by Scheme I is used. It has been found that the reactivity of the BIOS-Na crystalline forms of the present invention is higher than the reactivity of BIOS-Na dehydrated according to prior art methods.

Thus, according to another aspect of the present invention there is provided a crystalline BIOS-Na Form A.

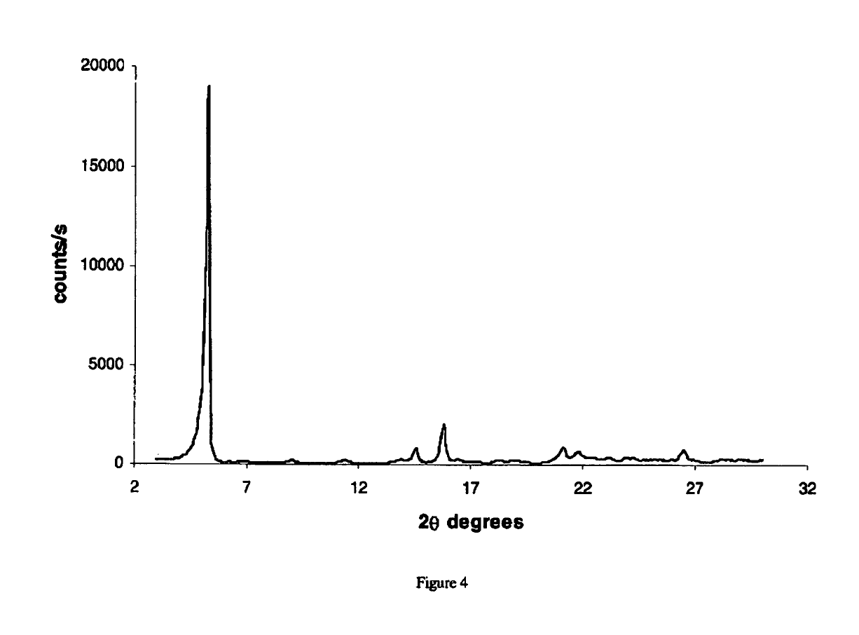

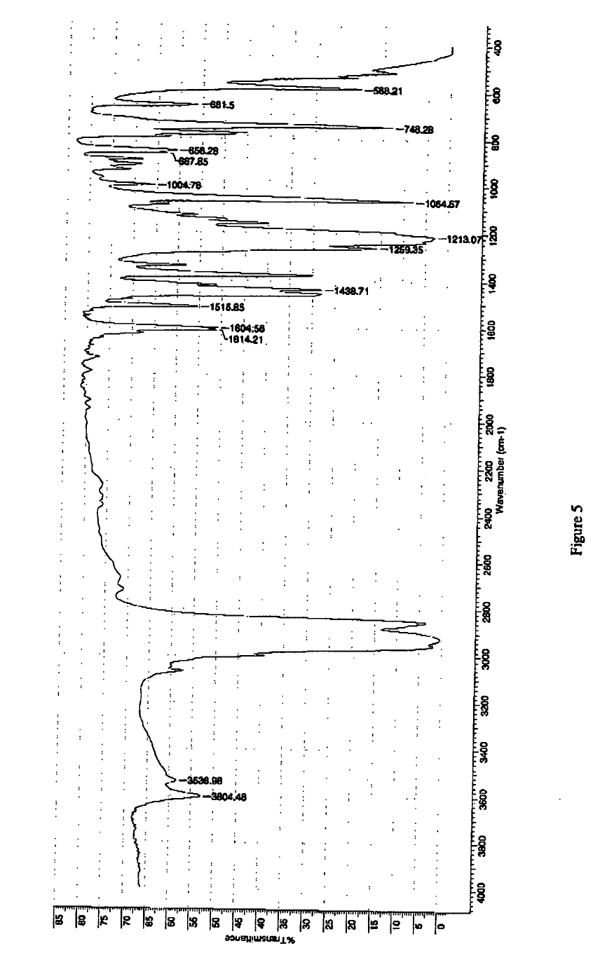

BIOS-Na Form A is characterized by a unique X-ray powder diffractogram, as presented in FIG. 4. The reflections. at 5.3, 6.9, 9.1, 11.5, 13.9, 14.6, 15.8, 16.5, 18.3, 21.2, 21.7, 23.2, 238, 24.2, 26.5, 28.2, 29.1 and 21.2±0.2 degrees 2θ are characteristic of BIOS-Na Form A. In Table 1, the position of peaks and the relative intensities of peaks of the powder X-ray diffractogram of BIOS-Na Form A are presented. BIOS-Na Form A is also characterized by a unique infrared (IR) spectrum, as presented in FIG. 5.

BIOS-Na Form A may be obtained by providing a mixture of BIOS-Na and toluene, refluxing the mixture while removing water therefrom, as described above with respect to the dehydration process, and isolating the thus formed BIOS-Na Form A from the mixture. Generally, BIOS-Na is mixed with toluene and heated to reflux temperature at which water is azeotropically distilled. When sufficiently dry, the mixture is allowed to cool leading to precipitation of fine crystals of BIOS-Na Form A, which can be isolated by, e.g., filtration,

BIOS-Na Form A can be further used as known in the art in the preparation of zonisamide. Substantially, an appropriate amount of BIOS-Na Form A is provided and is converted to zonisamide, for example according to the process described herein.

TABLE 1
BIOS-Na Form A-Powder X-ray diffraction
peak positions and intensities

| Relative | Peak | Relative | Peak |
|---|---|---|---|
| Intensity | Position | Intensity | Position |
| (%) | (2θ deg) | (%) | (2θ deg) |
| 100 | 5.3 | 1 | 21.2 |
| 1 | 6.9 | 4 | 21.7 |
| 1 | 9.1 | 3 | 23.2 |
| 1 | 11.5 | 2 | 238 |
| 1 | 13.9 | 2 | 24.2 |
| 4 | 14.6 | 2 | 26.5 |
| 10 | 15.8 | 4 | 28.2 |
| 1 | 16.5 | 1 | 29.1 |
| 1 | 18.3 | 1 | 21.2 |

Further according to the present invention there is provided crystalline BIOS-Na Form B.

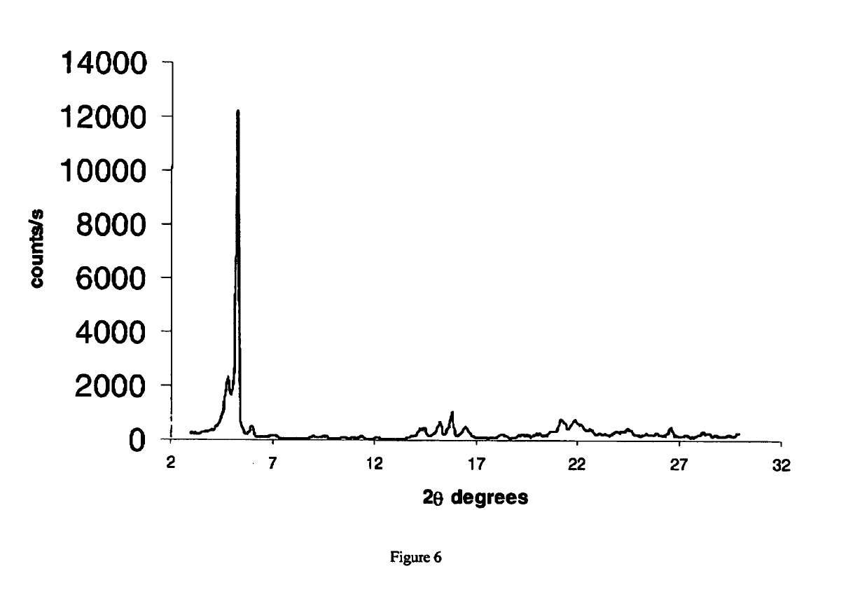

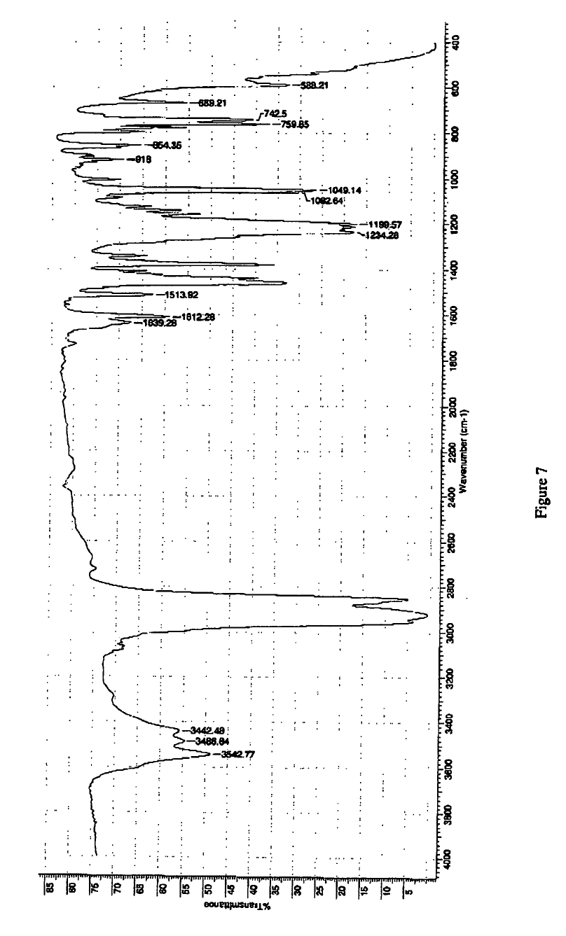

BIOS-Na Form B is characterized by a unique powder X-ray diffractogram, as is presented in FIG. 6. The reflections at 4.8, 5.3, 6.0, 7.1, 14.4, 15.3, 15.9, 16.5, 18.2, 19.4, 20.1, 21.2, 21.9, 22.2, 22.7, 23.9, 24.2, 24.5, 25.3, 25.9, 26.6, 27.3, 28.2, 28.5 and 29.9±0.2 degrees 2θ are characteristic of BIOS-Na Form B. In Table 2, the position of peaks and relative intensities of peaks of the powder X-ray diffractogram of BIOS-Na Form B arc presented. BIOS-Na Form B is also characterized by a unique infrared (IR) spectrum, as presented in FIG. 7.

BIOS-Na Form B may be obtained by providing a mixture of BIOS-Na in a solvent mixture of toluene and DMF, refluxing the mixture while removing water therefrom, as described above with respect to the dehydration process, and isolating the thus formed BIOS-Na Form B from the mixture. Generally, BIOS-Na is mixed with toluene and DMF and the mixture is heated to reflux temperature at which water is azeotropically distilled. When sufficiently dry, the mixture is allowed to cool leading to precipitation of crystals of BIOS-Na Form B, which can be isolated by, e.g., filtration.

The solvent mixture containing toluene :and IDMF typically includes from 5% to 10% (volume/volume) DMF relative to toluene, preferably from 6% to 7% (volume/volume) DMF relative to toluene.

Further preferably, the mixture contains at least 2 ml (or, e.g., at least 5 ml or at least 7 ml or at least 10 ml ) toluene for every gram sodium 1,2-benzisoxazole-3-methanesulfonate at least 0.1 mole (and preferably at least 1 mole) DMF for every mole sodium 1,2-benzisoxazole-3-methanesulfonate.

BIOS-Na Form B can be further used as known in the art in the preparation of zonisamide. Substantially, an appropriate amount of BIOS-Na Form A is provided and is convert to zonisamide, for example according to the process described herein.

TABLE 2
BIOS-Na Form B-Powder X-ray diffraction
peak positions and intensities

| Relative | Peak |
|---|---|
| Intensity | Position |
| (%) | (2θ deg) |
| 19 | 4.8 |
| 100 | 5.3 |
| 4 | 6.0 |
| 2 | 7.1 |
| 1 | 8.9 |
| 1 | 9.6 |
| 1 | 10.5 |
| 1 | 11.4 |
| 1 | 12.3 |
| 3 | 14.4 |
| 5 | 15.3 |
| 9 | 15.9 |
| 4 | 16.5 |
| 2 | 18.2 |
| 2 | 19.4 |
| 2 | 20.1 |
| 6 | 21.2 |
| 6 | 21.9 |
| 5 | 22.2 |
| 4 | 22.7 |
| 3 | 23.9 |
| 3 | 24.2 |
| 4 | 24.5 |
| 2 | 25.3 |
| 2 | 25.9 |
| 4 | 26.6 |
| 2 | 27.3 |
| 3 | 28.2 |
| 3 | 28.5 |
| 2 | 29.9 |

Both crystalline forms of BIOS-Na described herein, namely BIOS-Na Form A and Form B are substantially dehydrated.

As used herein, the phrase “substantially dehydrated” refers to substances that include a reduced water content, typically as low as 2% and preferably lower than 1.5%. As is demonstrated in the Examples section that follows, both BIOS-Na Form A and Form B were characterized as having a water content of about 1.3%.

As used herein throughout, the term “about” refers to ±10%.

Any of the crystalline Forms of BIOS-Na described herein can thus be efficiently used as novel intermediates for preparing zonisamide. Other dehydrated BIOS-Na, prepared by the process of dehydrating BIOS-Na described above, can also be beneficially used in the preparation of zonisamide, as discussed hereinabove.

Thus, according to fiber aspects of the present invention there is provided a process of preparing zonisamide, which is effected by providing any of the crystalline forms of BIOS-Na described herein, namely BIOS-Na Form A and BIOS-Na Form B and converting the crystalline or dehydrated BIOS-Na to zonisamide, using any of the procedures known in the art.

Converting the crystalline or dehydrated BIOS-Na described herein to zonisamide is preferably effected, according to the present embodiments, by providing a mixture of the selected BIOS-Na in a solvent and reacting the mixture with a chlorinating agent, so to provide the intermediate BIOS-Cl.

The dehydrated BIOS-Na (whether directly from the dehydration reaction or after isolation as crystalline BIOS-Na Form A or BIOS-Na Form B) is converted to 1,2-benzisoxazole-3-methanesulfonyl chloride (Compound IV, BIOS-Cl) by reaction with a chlorinating agent, such as POCl₃, SOCl₂, PCl₅ and oxalyl chloride, preferably oxalyl chloride. Preferably, the chlorination is performed under inert atmosphere such as nitrogen atmosphere.

When the chlorination reaction is performed directly on the product of the dehydration reaction, a BIOS-Na/solvent mixture generally already exists. When an isolated crystalline BIOS-Na is chlorinated, generally a first step is to make a mixture of BIOS-Na and a solvent. The process is preferably performed in an organic solvent and more preferably in an inert organic solvent. Non-limiting examples of suitable organic solvents include, but are not limited to, diethyl ether, diisopropyl ether, methyl tert-butyl ether, dichloromethane, chloroform, o-xylene, m-xylene, p-xylene, toluene and any mixture thereof. The most preferred solvent includes toluene, preferably substantially consisting of toluene (which forms a suspension).

Preferably, at least 7 volumes of the solvent (relative to the BIOS-Na) are used. More preferably, about 10 volumes of the solvent relative to the BIOS-Na are used.

Generally at least 1 molequivalent (to the amount of BIOS-Na) and no more than 1.2 molequivalents of a chlorinating agent, are used. Preferably, between 1.02 and 1.1 molequivalents of the chlorinating agents are used.

The chlorinating agent is preferably added portionwise (e.g., dropwise) over a period of time. During the time when the chlorinating agent is added, the temperature of the mixture is preferably between about 0° C. and 50° C., more preferably between about 10° C. and 20° C., more preferably to about 10° C. Subsequent to the addition of all the chlorinating agent, the temperature of the mixture is preferably maintained at a temperature of between about 0° C. and 50° C., preferably at least about 30° C., more preferably at least 40° C.

The chlorination reaction is preferably performed is the presence of a reaction facilitator. As used herein, the phrase “reaction facilitator” refers to a substance that promotes and/or facilitates a chemical reaction.

Known reaction facilitators of such chlorination reactions include organic amides, such as N,N-dimethylacetamide, N-methylpyrrolidone and N,N-dimethylformamide. Preferably, the reaction facilitator is N,N-dimethylformamide (DMF). It is thus preferred that the mixture includes a reaction facilitator. Thus, except in cases where sufficient DMF is already present in the mixture to act as an organic facilitator, it is preferred to add a reaction facilitator to the chlorination mixture. Preferably, at least 0.01 molequivalent of the reaction facilitator relative to the BIOS-Na are used. More preferably, about 0.1 molequivalent of the reaction facilitator relative to the BIOS-Na are used. The reaction facilitator is added to the mixture before, together with, during or after the addition of the chlorinating agent. Preferably, the reaction facilitator is added before the addition of any chlorinating agent.

The above procedure converts BIOS-Na to BIOS-Cl substantially quantitatively.

The thus-produced BIOS-Cl in the reaction solution is directly amidated, preferably without intermediate steps, by contact with ammonia to yield zonisamide (Compound I).

The ammonia used in the amidation reaction can be, for example, anhydrous, gaseous ammonia, aqueous ammonia or “masked ammonia”.

When aqueous ammonia is used, the amidation reaction is carried out in a biphasic system, which contains an aqueous phase that includes the aqueous ammonia and a water-immiscible solvent phase such as toluene.

When “masked ammonia” is used in the amidation reaction, the ammonia can be provided as, for example, an ammonium salt including ammonium carbonate, ammonium acetate, and ammonium formate.

Preferably, the amidation reaction is carried out with gaseous ammonia, which is bubbled into the reaction mixture. The gaseous ammonia can be anhydrous or non-anhydrous, with anhydrous ammonia being preferred. Nevertheless, it has been found (data not shown) that using non-anhydrous conditions in the amidation reaction (e.g., 300 ppm water content in the solvent), also resulted in efficient conversion of the BIOS-Cl to zonisamide.

Preferably, the reaction with gaseous ammonia is performed by bubbling ammonia gas through the reaction solution to make an amidation solution. Generally, at least about 2 equivalents, preferably 2.5 equivalents and even more preferably 4 equivalents of ammonia gas (relative to a quantitative yield of the BIOS-Cl), either anhydrous or non-anhydrous ammonia gas, are bubbled through the solution. It is preferred that the ammonia gas is bubbled through the solution for at least one hour and preferably for at least two hours.

Preferably, the reaction solution is cooled prior to the addition of the ammonia, preferably to about 10° C. During the bubbling procedure the temperature of the amidation solution is between about 0° C. and 30° C., preferably between about 10° C. and 18° C.

As the amidation of the BIOS-Cl proceeds, a zonisamide-containing precipitate is formed. When the amidation of the BIOS-Cl is completed, the zonisamide-containing precipitate is isolated, for example by filtering. The thus-isolated zonisamide-containing precipitate is of very high purity typically greater than. 98% zonisamide and even greater than 99% zonisamide, as determined by HPLC.

The zonisamide-containing precipitate can be further purified, so as to provide highly pure zonisamide. Purification can be effected by any of the known purification methods, including but not limited to, extraction, column chromatography, preparative low-pressure liquid chromatography, preparative high-pressure liquid chromatography, re-crystallization, slurrying and any combination thereof.

According to a preferred embodiment of this aspect of the present invention, zonisamide is purified by slurrying and/or recrystallization, as is detailed herein. Preferably, zonisamide is purified by slurrying followed by recrystallization.

A preferred manner of purifying the zonisamide is by slurrying the zonisamide-containing precipitate in water or an aqueous solution.

Although the slurrying is preferably performed in water, more preferably the slurrying is performed in an aqueous solution of an inorganic base. The inorganic base can be, for example, ammonium hydroxide, sodium hydroxide, potassium hydroxide and the like, and is preferably ammonium hydroxide.

A solution containing at least 5% ammonium hydroxide, more preferably at least 10%, more preferably at least 20%, and most preferably 25% ammonium hydroxide is used in the slurrying procedure.

At least 2 ml to 1 gram of zonisamide-containing precipitate are preferably used in the slurrying procedure. More preferably, about 5.5 ml to 1 gram of the zonisamide-containing precipitate are used.

The slurrying process is preferably carried out at ambient temperature, during at least 1 hour and more preferably during 2 hours.

The purified zonisamide is then isolated from the slurry, for example, by filtering.

Recrystallization of zonisamide is performed using known procedures, preferably from an alcohol/water mixture. The alcohol/water ratio, in a recrystallization mixture, preferably ranges from 10:1 to 1:10. In a preferred embodiment, the ratio is about 1:7 alcohol/water.

The alcohol used in the recrystallization procedure is preferably a water-miscible alcohol, that is methanol, ethanol, isopropanol or n-propanol.

Using the above purification procedure and the above process, highly pure zonisamide is obtained.

The process described herein for preparing zonisamide utilizes dehydrated and/or crystalline BIOS-Na, as described herein. However, as is demonstrated in the Examples section that follows, this process can be efficiently effected by preparing the dehydrated and/or crystalline forms of BIOS-Na in situ, such that the formed dehydrated intermediate can be directly further reacted to produce zonisamide in a one-pot process. This procedure is highly efficient since it enables the in situ dehydration of the reacting solution and thus circumvents the need to isolate and dry the intermediate used in the press prior to the reaction.

Such a process can thus be advantageously utilized for preparing zonisamide from any suitable intermediate thereof.

Thus, according to an additional aspect of the present invention there is provided another process of preparing zonisamide, which is effected by:

providing a mixture of a zonisamide intermediate and a solvent;

refluxing the mixture while removing water therefrom (as an azeotrope, as is detailed hereinabove), and thus dehydrating the zonisamide intermediate and optionally also the solvent, by means of azeotropic distillation (as is described hereinabove), so as to provide a mixture containing a dehydrated zonisamide intermediate and preferably also a dehydrated solvent; and

converting the dehydrated zonisamide intermediate to zonisamide.

The process according to this aspect of the present invention can be applied with any suitable intermediate of zonisamide, including, for example, BIOS-Na and any crystalline form thereof, and other salts of 1,2-benzisoxazole-3-methanesulfonic acid (e.g., BIOS-Ba, BIOS-Ca and the like, as described, for example, in WO 03/020708, which is incorporated by reference as if fully set forth herein) and any crystalline form thereof.

Other zonisamide intermediates that can be beneficially used for preparing zonisamide according to this aspect of the present invention include, for example, BIOS-Cl (using the process described, for example, in WO 03/072552, which is incorporated by reference as if fully set forth herein) and BIOS-H.

Additional intermediates that can be beneficially used for preparing zonisamide according to this aspect of the present invention include, for example. BIOS-NH₄ and a 1,2-benzisoxazole-3-methanesulfonic acid ester, described, for example, in U.S. Provisional Patent applications Nos. 60/582,360 and 60/622,029, and in a U.S. Patent Application entitled “Derivatives of 1,2-benzioxazole-3-methane sulfonic acid as novel intermediates for the preparation of zonisamide”, which is co-filed with the instant application, the contents of which are incorporated herein id their entirety.

Converting the dehydrated intermediate to zonisamide can be effected by using any of the known procedures, depending on the selected zonisamide intermediate.

Thus, for example, in cases where the intermediate is a salt of 1,2-benzisoxazole-3-methanesulfonic acid (e.g., BIOS-Na, BIOS-Ca, BIOS-Ba or BIOS-NH₄) or a 1,2-benzisoxazole-3-methanesulfonic acid ester, converting the intermediate to zonisamide can be effected by reacting the mixture with a chlorinating agent, so as to provide BIOS-Cl; reacting the BIOS-Cl with ammonia, so as to provide zonisamide; and isolating the zonisamide. Preferred features of such a conversion are described hereinabove.

The solvent used in the process according to this aspect of the present invention can be, for example, a non-polar solvent such as o-xylene, m-xylene, p-xylene, toluene, diethyl ether, diisopropyl ether, methyl tert-butyl ether, dichloromethane, chloroform, and any mixture thereof, a polar solvent such as DMF, N-methyl pyrrolidone and NN-dimethylacetamide, and any mixture thereof; or a mixture of a polar solvent and a non-polar solvent.

Preferably, the solvent comprises toluene. Further preferably, the solvent comprises a mixture of toluene and DMF.

Adding DMF to the mixture containing the intermediate is particularly beneficial since, as described above, DMF can be used as a reaction facilitator in the chlorination reaction. Thus, adding DMF to the mixture containing the intermediate is advantageous since (i) DMF is thus dehydrated in situ and its dehydration prior to addition to the reaction mixture is avoided; and (ii) the addition of DNE at a later stage of the process is circumvented.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

### Experimental Methods

A prior art crystalline form of BIOS-Na monohydrate as well as BIOS-Na Forms A and B of the present invention were characterized by powder X-ray diffraction, which produces a fingerprint of a particular crystalline form. Measurements of 2θ values typically are accurate to within ±0.2 degrees 2θ. X-ray diffraction data were acquired using a PHILIPS X-ray diffractometer model PW 1050-70. System description: K_{α1}=1.54178 Å, voltage 40 kV, current 28 mA, diversion slit=1°, receiving slit=0.2 mm, scattering slit=1° with a Graphite monochromator. Experiment parameters: pattern measured between 2θ=40° and 2θ=30° with 0.05° increments; count time was 0.5 second per increment

The crystalline BIOS-Na Forms A and B of the present invention were further characterized by Fourier-transform infrared spectroscopy (FTIR) to an accuracy of ±4 cm⁻¹ using a Nicolet Fourier-transform infrared spectrometer model Avatar 360, with Omnic software version 5.2. All samples were run as KBr pellets. FTIR is a well-known spectroscopy analysis in which absorption of IR energy by the sample results from transitions between molecular vibrational energy levels. FTIR is used, in modem practice, mainly for identification of functional groups in the molecule. However, different polymorphic forms also show differences in FTIR.

The prior art crystalline form of BIOS-Na monohydrate was also characterized by differential scanning calorimetry (DSC), run on TA instruments model Q1000, with Universal software version 3.88. Samples were analyzed inside crimped 40 μl Aluminum pans. Heating rate for all samples was 5° C./min.

Differential scanning calorimetry (DSC) graphs were recorded using a TA Instruments Q1000 Thermal Analyzer with Universal software (version 3.88). Samples were analyzed inside crimped 40 μl Aluminum pans at a heating rate of 5° C./min.

The prior art crystalline form of BIOS-Na monohydrate was also characterized by thermogravimetric analysis (TGA), a measure of the thermally induced weight loss of a material as a function of the applied temperature. Thermogravimetric analysis (TGA) was performed using a TA Instruments Q500 Thermal Analyzer with Universal Software (version 3.88). Samples were analyzed inside platinum baskets at heating rate of 5° C./minute.

The water content of the prior art crystalline form of BIOS-Na monohydrate as well as BIOS-Na Forms A and B of the present invention were measured using a Karl-Fischer titration. The Karl Fisher assay for determining water content is well known to one skilled in the art and is described in Pharmacopeia form, Vol. 24, No. 1, p.5438 (January-February 1998). Water content was measured using a Karl Fischer Titrator (Mettler Toledo Model DL-53) according to standard procedures.

HPLC measurements were performed using HPLC JASCO, LC-1500 series, equipped with Inertsil ODS-2, 5 μm, 4.6×25 cm column, and a UV detector operated on 238 nm. Analyses were performed using the following mobile phase: 0.08 M tetrabutylammonium hydroxide buffer at pH 8.0 with H₃PO₄ (70%), acetonitrile (25 %) and methanol (5%), at a flow rate of 1.0 ml/minute.

### Reference Example 1

### Preparation of BIOS-Na According to Example 1 of U.S. Pat. No. 4,172,896.

BIOS-Na (Compound III) was prepared in accordance with the method described in Example 1 of U.S. Pat. No. 4,172,896.

A solution of sodium sulfite (24.3 grams) in water (390 ml) was added to a solution of 3-bromomethyl-1,2-benzisoxazole (24 grams, Compound II) in methanol (390 ml), stirred with heating to 50° C. for 4 hours. After completion of the reaction, the solution was concentrated under reduced pressure. The resulting crystalline residue was heated to about 50-60° C. in methanol (750 ml) and the solution filtered. The clear filtrate was concentrated under reduced pressure and the resulting crystalline residue was washed with diethyl ether to give crystalline sodium 1,2-benzisoxazole-3-methanesulfonate (18 grams, BIOS-Na, Compound III).

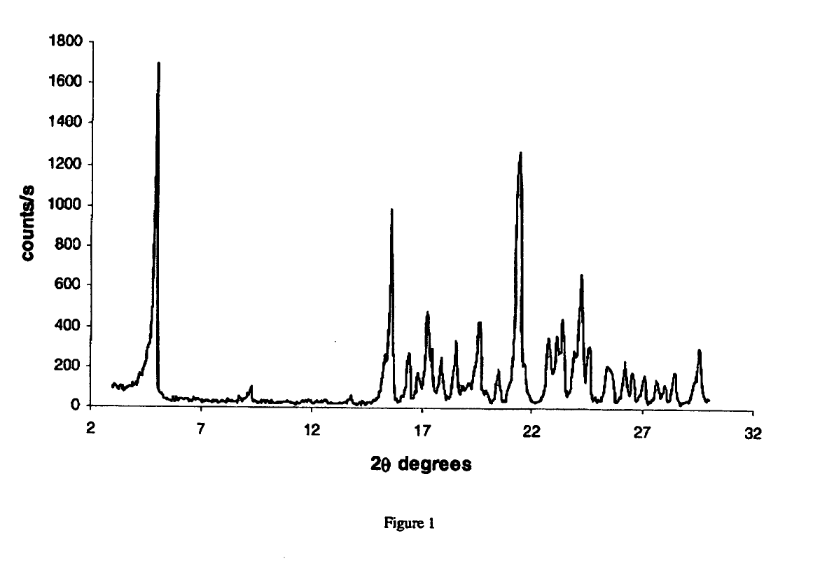

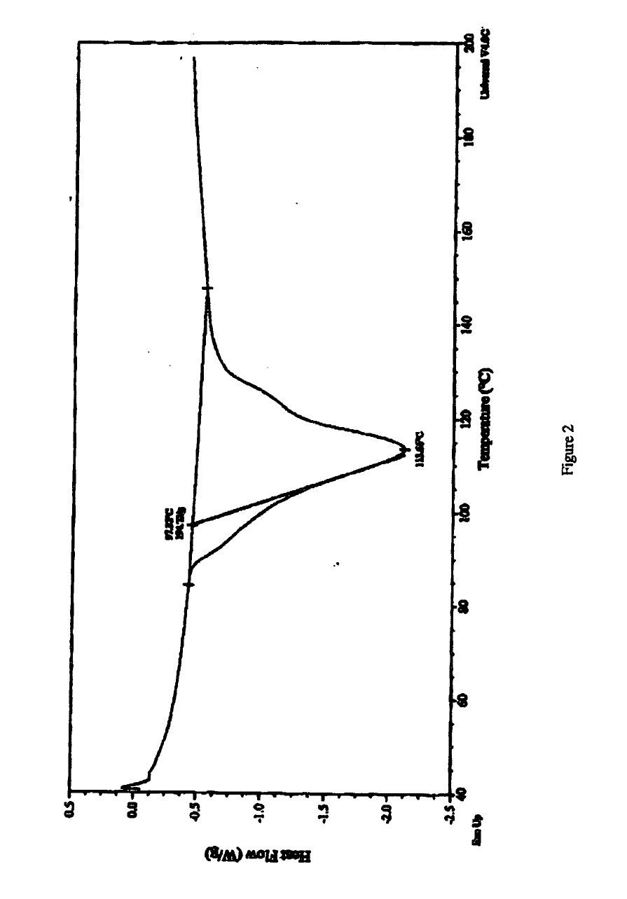

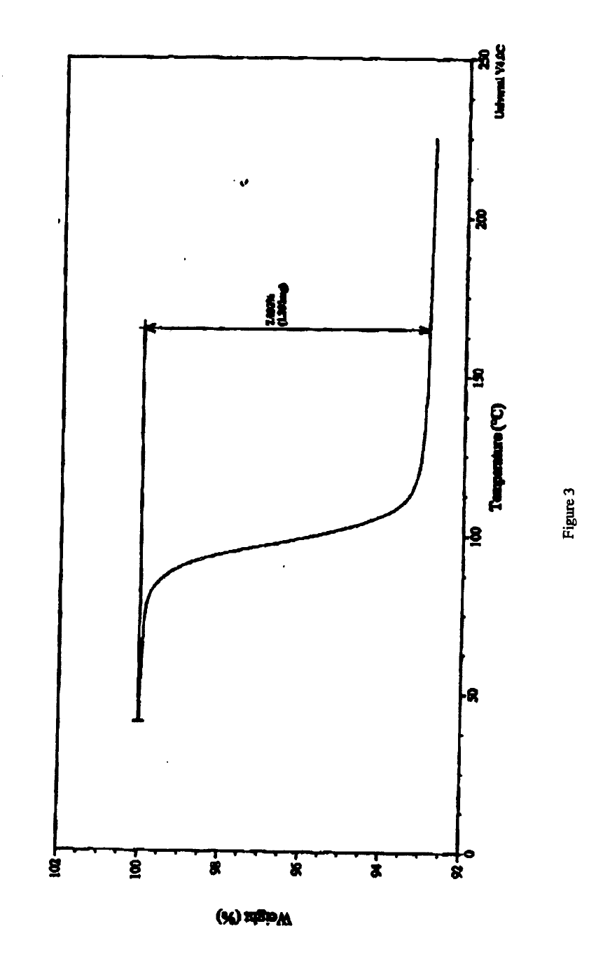

Thc thus-produced BIOS-Na was analyzed using X-Ray powder diffraction (results depicted in FIG. 1), differential scanning calorimetry (results depicted in FIG. 2) and thermal gravimetry (results depicted in FIG. 3).

The water content of the thus-produced BIOS-Na was 7% as measured by Karl-Fischer titration, indicating that the crystals were of BIOS-Na monohydrate.

### Example 1

### Preparation of BIOS-Na Form A

A 500 ml three-necked flask, equipped with mechanical stirrer, Dean-Stark trap and condenser, was charged with BIOS-Na monohydrate (20 grams, prepared as described in Reference Example 1 above) and toluene (220 ml). The resulting suspension was heated to reflux so that water was azeotropically distilled during 4 hours.

The solution was cooled under nitrogen atmosphere to ambient temperature and the resulting crystalline precipitate was collected by filtration under nitrogen atmosphere to yield BIOS-Na Form A (18.3 grams, 98.6% yield).

The thus-produced BIOS-Na was analyzed using X-Ray powder diffraction (results depicted in FIG. 4) and infrared spectrometry (results depicted in FIG. 5).

The water content of the thus-produced BIOS-Na Form A was 1.3% as measured by Karl-Fischer titration. Example 2

### Preparation of zonisamide from BIOS-NA Form A

A 500 ml three necked flask, equipped with mechanical stirrer, Dean-Stark trap and condenser, was charged with BIOS-Na monohydrate (20 grams, prepared as described in Reference Example 1 above) and toluene (220 ml). The resulting suspension was heated to reflux so that water was azeotropically distilled during 4 hours.

The resulting suspension was cooled under nitrogen atmosphere to 10° C. and DMF (6.4 ml, 1.04 equivalents) was added into the mixture. Oxalyl chloride (10.5 grams, 1.04 equivalents) was added dropwise to the mixture at 10-15° C. during 1 hour and the reaction mixture heated to 40° C. for 1 hour. Ammonia gas (5.5 grams, 4 equivalents) was bubbled into the reaction mixture at 10-18° C. during 2 hours. The resulting precipitate was collected by filtration and slurried in water (80 ml) at ambient temperature for 2 hours. A solid was collected from the slurry by filtration, washed with water and dried under reduced pressure at 50° C. overnight to obtain crude zonisamide (15.6 grams) in 93% yield, (purity by HPLC: 99.05%).

The crude zonisamide (15.6 grams) was dissolved with heating in a mixture of methanol (150 ml) and water (20 ml) and the solution was thereafter filtered. A portion of the methanol (about 65 ml) was distilled from the filtrate. The residual solution was cooled to ambient temperature and maintained at 10-15° C. for 16 hours. The resulting colorless crystals were collected by filtration, washed with a cool mixture of water and methanol and dried under reduced pressure at 50° C. overnight to obtain pure zonisamide (12.6 grams) in 75% overall yield (purity by HPLC: 99.94

### Example 3

### Preparation of BIOS-Na Form B

A 500 ml three-necked flask, equipped with mechanical stirrer, Dean-Stark trap and condenser, was charged with BIOS-Na monohydrate (20 grams, prepared as described in Reference Example 1 above), toluene (100 ml) and DMF (6.4 ml, 1.04 equivalents). The resulting suspension was heated to 70° C. to form a jelly-like mixture. The resulting jelly-like mixture was heated to reflux so that water was azeotropically distilled during 2 hours. Toluene (100 ml) was added and the reflux with azeotropic distillation continued for 2 hours.

The resulting suspension was cooled under nitrogen atmosphere to ambient temperature. The precipitate was collected by filtration and washed with n-hexane to yield BIOS-Na Form B (18.5 grams).

The thus-produced BIOS-Na was analyzed using X-Ray powder diffraction (results depicted in FIG. 6) and infrared spectrometry (results depicted in FIG. 7).

The water content of the thus-produced BIOS-Na Form B was 1.3% as measured by Karl-Fischer titration.

### Example 4

### Preparation of zonisamide from BIOS-Na Form B

A 500 ml three-necked flask, equipped with mechanical stirrer, Dean-Stark trap and condenser, was charged with BIOS-Na monohydrate (20 grams, prepared as described in Reference Example 1 above), toluene (100 ml) and DMF (6.4 ml, 1.04 equivalents). The resulting suspension was heated to 70° C. forming a jelly-like mixture. The resulting jelly-like mixture was heated to reflux so that water was azeotropically distilled during 2 hours. Toluene (100 ml) was added and the reflux with azeotropic distillation continued for 2 hours

The suspension was cooled under nitrogen atmosphere to 10° C. Oxalyl chloride (10.5 rams, 1.04 equivalents) was added dropwise to the mixture at 10-15° C. during 1 hour and the reaction mixture was heated to 40° C. for 0.5 hour. Ammonia gas (5.5 grams, 4 equivalents) was then bubbled into the reaction mixture at 10-18° C. during 2 hours.

The resulting precipitate was collected by filtration and slurried in water (80 ml) at ambient temperature for 2 hours. A solid was collected from the slurry by filtration, washed with water and dried under reduced pressure at 50° C. overnight to obtain crude zonisamide (14.8 grams) in 88.8% yield (purity by HPLC: 98.53%).

The crude zonisamide (14.9grams) was dissolved with heating in a mixture of methanol (140 ml) and water (15 ml) and the solution was thereafter filtered. A portion of the methanol (about 60 ml) was distilled from the filtrate. The residual solution was cooled to ambient temperature and maintained at 10-15° C. for 16 hours. The resulting colorless crystals were collected by filtration, washed with a cool mixture of water and methanol and dried under reduced pressure at 50° C. overnight to obtain pure zonisamide (12.5 grams) in 74.5% overall yield (purity by HPLC: 99.95

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will bc apparent to those skilled in the art. Accordingly, the present invention is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A crystalline sodium 1,2-benzisoxazole-3-methanesulfonate Form A.

2. The crystalline sodium 1,2-benzisoxazole-3-methanesulfonate Form A of claim 1, comprising at least one of the characteristics selected from the group consisting of:
a powder X-ray diffraction pattern exhibiting peaks at diffraction angles of about 5.3 and 15.8±0.2 °2θ; and
an infrared spectrum exhibiting absorption peaks at 3605, 3532, 1620, 1605, 1064 and 662 cm⁻¹.

3. The crystalline sodium 1,2-benzisoxazole-3-methanesulfonate Form A of claim 2, wherein said powder X-ray diffraction pattern further exhibits peaks at diffraction angles of about 14.5, 21.1, 21.8 and 26.4±0.2 °2θ.

4. The crystalline sodium 1,2-benzisoxazole-3-methanesulfonate Form A of claim 2, wherein said infrared spectrum further exhibits absorption peaks at 3605, 3532, 2924, 2853, 1620, 1605, 1516, 1438, 1341, 1260, 1213, 1160, 1064, 1005, 777, 748, 662, 588 and 521 cm⁻¹.

5. The crystalline sodium 1,2-benzisoxazole-3-methanesulfonate Form A of claim 2, wherein said powder X-ray diffraction pattern is substantially as depicted in FIG. 4.

6. The crystalline sodium 1,2-benzisoxazole-3-methanesulfonate Form A of claim 2, wherein said infrared spectrum is substantially as depicted in FIG. 5.

7. The crystalline sodium 1,2-benzisoxazole-3-methanesulfonate Form A of claim 1, having a water content lower than 1.5%.

8. The crystalline sodium 1,2-benzisoxazole-3-methanesulfonate Form A of claim 7, having a water content that ranges from about 1% to about 1.4%.

9. A process of preparing the crystalline sodium 1,2-benzisoxazole-3-methanesulfonate Form A of claim 1, the process comprising:
providing a mixture including sodium 1,2-benzisoxazole-3-methanesulfonate and toluene;
refluxing said mixture while removing water therefrom, and
isolating the sodium 1,2-benzisoxazole-3-methanesulfonate Form A from said mixture, thereby obtaining the crystalline sodium 1,2-benzisoxazole-3-methanesulfonate Form A.

10. The process of claim 9, further comprising, subsequent to said isolating, drying the sodium 1,2-benzisoxazole-3-methanesulfonate Form A.

11. A process of preparing 1,2-benzisoxazole-3-methanesulfonamide (zonisamide), the process comprising:
providing the crystalline sodium 1,2-benzisoxazole-3-methanesulfonate Form A of claim 1; and
converting said sodium 1,2-benzisoxazole-3-methanesulfonate Form A to 1,2-benzisoxazole-3-methanesulfonamide (zonisamide).

12. A crystalline sodium 1,2-benzisoxazole-3-methanesulfonate Form B.

13. The crystalline sodium 1,2-benzisoxazole-3-methanesulfonate Form B of claim 12 comprising at least one of the characteristics selected from the group consisting of:
a powder X-ray diffraction pattern exhibiting peaks at diffraction angles of about 4.8 and 5.3±0.2 °2θ; and
an infrared spectrum exhibiting absorption peaks at 3543, 3484, 3436, 1639, 1613, 1049, 761 and 742 cm⁻¹.

14. The crystalline sodium 1,2-benzisoxazole-3-methanesulfonate Form B of claim 13, wherein said powder X-ray diffraction pattern further exhibits peaks at diffraction angles of about 6.0, 14.4, 15.2, 15.7, 16.4, 21 .1, 21.8 and 26.5±0.2 °2θ.

15. The crystalline sodium 1,2-benzisoxazole-3-methanesulfonate Form B of claim 13, wherein said infrared spectrum further exhibits absorption peaks at 3543, 3484, 3436, 2923, 2854, 1639, 1613, 1514, 1439, 1342, 1235, 1212, 1199, 1160, 1063, 1049, 1006, 761, 742, 669 and 589 cm⁻¹.

16. The crystalline sodium 1,2-benzisoxazole-3-methanesulfonate Form B of claim 13, wherein said powder X-ray diffraction pattern is substantially as depicted in FIG. 6.

17. The crystalline sodium 1,2-benzisoxazole-3-methanesulfonate Form B of claim 13, wherein said infrared spectrum is substantially as depicted in FIG. 7.

18. The crystalline sodium 1,2-benzisoxazole-3-methanesulfonate Form B of claim 12, having a water content lower than 1.5%.

19. The crystalline sodium 1,2-benzisoxazole-3-methanesulfonate Form B of claim 18, having a water content that ranges from about 1% to about 1.4%.

20. A process of preparing the crystalline sodium 1,2-benzisoxazole-3-methanesulfonate Form B of claim 12, the process comprising:
providing a mixture including sodium 1,2-benzisoxazole-3-methanesulfonate and a solvent containing toluene and N,N-dimethylformamide (DMF) as solvent components;
refluxing said mixture while removing water therefrom; and
isolating the sodium 1,2-benzisoxazole-3-methanesulfonate Form B from said mixture, thereby obtaining the crystalline sodium 1,2-benzisoxazole-3-methanesulfonate Form B.

21. The process of claim 20, wherein the mixture comprises of at least 0.1 molequivalent of DMF relative to sodium 1,2-benzisoxazole-3-methanesulfonate.

22. The process of claim 20, further comprising, subsequent to said isolating, drying the sodium 1,2-benzisoxazole-3-methanesulfonate Form B.

23. A use of sodium 1,2-benzisoxazole-3-methanesulfonate Form B of claim 12 in the preparation of 1,2-benzisoxazole-3-methanesulfonamide (zonisamide).

24. A process of preparing 1,2-benzisoxazole-3-methanesulfonamide (zonisamide), the process comprising:
providing the crystalline sodium 1,2-benzisoxazole-3-methanesulfonate Form B of claim 12; and
converting said sodium 1,2-benzisoxazole-3-methanesulfonate Form B to 1,2-benzisoxazole-3-methanesulfonamide (zonisamide).

25. A process of dehydrating sodium 1,2-benzisoxazole-3-methanesulfonate, the process comprising:
providing a mixture of said sodium 1,2-benzisoxazole-3-methanesulfonate and a non-aqueous solvent; and
refluxing said mixture while removing water.

26. The process of claim 25, wherein said solvent is selected from the group consisting of toluene, DMF and a mixture thereof.

27. A process of preparing zonisamide, the process comprising:
providing a mixture containing a zonisamide intermediate and a solvent;
refluxing said mixture while removing water therefrom; and
converting said zonisamide intermediate to zonisamide, thereby obtaining the zonisamide.

28. The process of claim 27, wherein said zonisamide intermediate is selected from the group consisting of a salt of 1,2-benzisoxazole-3-methanesulfonic acid, 1,2-benzisoxazole-3-methanesulfonic acid, 1,2-benzisoxazole-3-methanesulfonic acid ammonium salt, a 1,2-benzisoxazole-3-methanesulfonic acid ester and 1,2-benzisoxazole-3-methanesulfonoyl chloride.

29. The process of claim 27, wherein said solvent is selected from the group consisting of diethyl ether, diisopropyl ether, methyl tert-butyl ether, dichloromethane, chloroform, o-xylene, m-xylene, p-xylene, toluene, DMF and any mixture thereof.

30. Thc process of claim 29, wherein said solvent is selected from the group consisting of toluene, DW and a mixture thereof.
